(19) United States
(12) Patent Application Publication
(10) Pub. No.: US 2023/0049775 A1
(43) Pub. Date: Feb. 16, 2023

(54) DEGRADABLE DELIVERY PARTICLES BASED FROM AMINE CONTAINING NATURAL MATERIALS

(71) Applicant: Encapsys,LLC, Appleton, WI (US)

(72) Inventor: Linsheng Feng, Menasha, WI (US)

(73) Assignee: Encapsys,LLC, Appleton, WI (US)

(21) Appl. No.: 17/819,205

(22) Filed: Aug. 11, 2022

Related U.S. Application Data
(60) Provisional application No. 63/232,713, filed on Aug. 13, 2021.
(60) Provisional application No. 63/287,883, filed on Dec. 9, 2021.

(51) Int. Cl. B01J 13/16

(52) U.S. Cl. CPC B01J 13/16

(57) ABSTRACT

An improved delivery particle comprising a benefit agent core material and a shell encapsulating the core material is described, along with a process for forming such a delivery particle and articles of manufacture. The shell is the reaction product of: i) an isocyanate or acid chloride or acrylate with ii) an amine-containing natural material having free amino moieties, and iii) an a, β -unsaturated compound, the a, β -unsaturated compound forming C-N covalent bonds with the amine moieties of the natural material. The delivery particle of the invention has improved release characteristics, with enhanced degradation characteristics in OECD test method 301B.

### FIELD OF THE INVENTION

This invention relates to capsule manufacturing processes and biodegradable delivery particles produced by such processes, the delivery particles containing a core material and a shell encapsulating the core.

### DESCRIPTION OF THE RELATED ART

Microencapsulation is a process where droplets of liquids, particles of solids or gasses are enclosed inside a solid shell and are generally in the micro-size range. The core material is separated from the surrounding environment by the shell. Microencapsulation technology has a wide range of commercial applications for different industries. Overall, capsules are capable of one or more of (i) providing stability of a formulation or material via the mechanical separation of incompatible components, (ii) protecting the core material from the surrounding environment, (iii) masking or hiding an undesirable attribute of an active ingredient and (iv) controlling or triggering the release of the active ingredient to a specific time or location. All of these attributes can lead to an increase of the shelf-life of several products and a stabilization of the active ingredient in liquid formulations.

Various processes for microencapsulation, and exemplary methods and materials are set forth in Schwantes (U.S. Pat. No. 6,592,990), Nagai et al. (U.S. Pat. No. 4,708,924), Baker et al. (U.S. Pat. No. 4,166,152), Wojciak (U.S. Pat. No. 4,093,556), Matsukawa et al. (U.S. Pat. No. 3,965,033), Ozono (U.S. Pat. No. 4,588,639), Irgarashi et al. (U.S. Pat. No. 4,610,927), Brown et al. (U.S. Pat. No. 4,552,811), Scher (U.S. Pat. No. 4,285,720), Jahns et al. (U.S. Pat. Nos. 5,596,051 and 5,292,835), Matson (U.S. Pat. No. 3,516,941), Foris et al. (U.S. Pat. Nos. 4,001,140; 4,087,376; 4,089,802 and 4,100,103), Greene et al. (U.S. Pat. Nos. 2,800,458; 2,800,457 and 2,730,456), Clark (U.S. Pat. No. 6,531,156), Hoshi et al. (U.S. Pat. No. 4,221,710), Hayford (U.S. Pat. No. 4,444,699), Hasler et al. (U.S. Pat. No. 5,105,823), Stevens (U.S. Pat. No. 4,197,346), Riecke (U.S. Pat. No. 4,622,267), Greiner et al. (U.S. Pat. No. 4,547,429), and Tice et al. (U.S. Pat. No. 5,407,609), among others and as taught by Herbig in the chapter entitled “Microencapsulation” in Kirk-Othmer Encyclopedia of Chemical Technology, V.16, pages 438-463.

Core-shell encapsulation is useful to preserve actives, such as benefit agents, in harsh environments and to release them at the desired time, which may be during or after use of goods incorporating the encapsulates. Among various mechanisms that can be used for release of benefit agent from the encapsulates, the one commonly relied upon is mechanical rupture of the capsule shell through friction or pressure. Selection of mechanical rupture as the release mechanism constitutes another challenge to the manufacturer, as rupture must occur at specific desired times, even if the capsules are subject to mechanical stress prior to the desired release time.

Industrial interest for encapsulation technology has led to the development of several polymeric capsules chemistries which attempt to meet the requirements of biodegradability, low shell permeability, high deposition, targeted mechanical properties and rupture profile. Increased environmental concerns have put the polymeric capsules under scrutiny, therefore manufacturers have started investigating sustainable solutions for the encapsulation of benefit agents.

Biodegradable materials exist and are able to form delivery particles via coacervation, spray-drying or phase inversion precipitation. However, the delivery particles formed using these materials and techniques are highly porous and not suitable for aqueous compositions containing surfactants or other carrier materials, since the benefit agent is prematurely released to the composition.

Non-leaky and performing delivery particles in aqueous surfactant-based compositions exist, however due to its chemical nature and cross-linking, they are not biodegradable.

Encapsulation can be found in areas as diverse as pharmaceuticals, personal care, textiles, food, coatings and agriculture. In addition, the main challenge faced in encapsulation is that a complete retention of the encapsulated active within the capsule is required throughout the whole supply chain, until a controlled or triggered release of the core material is applied. There are significantly limited microencapsulation technologies that can fulfill the rigorous criteria for long-term retention and active protection capability for commercial needs, especially when it comes to encapsulation of small molecules.

Delivery particles are needed that are biodegradable yet have high structural integrity so as to reduce leakage and resist damage from harsh environments.

### DEFINITIONS

As used herein, reference to the term “(meth)acrylate” or “(meth)acrylic” is to be understood as referring to both the acrylate and the methacrylate versions of the specified monomer, oligomer and/or prepolymer, (for example “isobornyl (meth)acrylate” indicates that both isobornyl methacrylate and isobornyl acrylate are possible, similarly reference to alkyl esters of (meth)acrylic acid indicates that both alkyl esters of acrylic acid and alkyl esters of methacrylic acid are possible, similarly poly(meth)acrylate indicates that both polyacrylate and polymethacrylate are possible). Similarly, the use of the phrase “prepolymer” means that the referenced material may exist as a prepolymer or combination of oligomers and prepolymers. Similarly, it is to be understood that the general reference herein to (meth)acrylate or (meth)acrylates, e.g., “water soluble (meth)acrylates”, “water phase (meth)acrylate”, etc., is intended to cover or include the (meth)acrylate monomers and/or oligomers. Additionally, the descriptors "water soluble or dispersible", water soluble", and "water dispersible" when referencing certain (meth)acrylate monomers and/or oligomers or initiators means that the specified component is soluble or dispersible in the given matrix solution on its own or in the presence of a suitable solubilizer or emulsifier or upon attainment of certain temperatures and/or pH.

Each alkyl moiety herein, unless otherwise indicated, can be from C₁ to C₈, or even from C₁ to C₂₄. Poly(meth)acrylate materials are intended to encompass a broad spectrum of polymeric materials including, for example, polyester poly(meth)acrylates, urethane and polyurethane poly(meth)acrylates (especially those prepared by the reaction of a hydroxyalkyl (meth)acrylate with a polyisocyanate or a urethane polyisocyanate), methyl cyanoacrylate, ethyl cyanoacrylate, diethylene glycol di(meth)acrylate, trimethylolpropane tri(meth)acrylate, ethylene glycol di(meth)acrylate, allyl (meth)acrylate, glycidyl (meth)acrylate, (meth)acrylate functional silicones, di-, tri- and tetraethylene glycol di(meth)acrylate, dipropylene glycol di(meth)acrylate, polyethylene glycol di(meth)acrylate, di(pentamethylene glycol) di(meth)acrylate, ethylene di(meth)acrylate, neopentyl glycol di(meth)acrylate, ethoxylated bisphenol A di(meth)acrylates, bisphenol A di(meth)acrylates, diglycerol di(meth)acrylate, tetraethylene glycol dichloroacrylate, 1,3-butanediol di(meth)acrylate, neopentyl di(meth)acrylate, polyethylene glycol di(meth)acrylate and dipropylene glycol di(meth)acrylate and various multifunctional (meth)acrylates and multifunctional amine (meth)acrylates. Monofunctional acrylates, i.e., those containing only one acrylate group, may also be advantageously used. Typical monoacrylates include 2-ethylhexyl (meth)acrylate, 2-hydroxyethyl (meth)acrylate, cyanoethyl (meth)acrylate, 2-hydroxypropyl (meth)acrylate, p-dimethyl aminoethyl (meth)acrylate, lauryl (meth)acrylate, cyclohexyl (meth)acrylate, tetrahydrofurfuryl (meth)acrylate, chlorobenzyl (meth)acrylate, amino alkyl(meth)acrylate, various alkyl(meth)acrylates and glycidyl (meth)acrylate. Of course, mixtures of (meth)acrylates or their derivatives as well as combinations of one or more (meth)acrylate monomers, oligomers and/or prepolymers or their derivatives with other copolymerizable monomers, including acrylonitriles and methacrylonitriles may be used as well. Multifunctional (meth)acrylate monomers will typically have at least two, at least three, and preferably at least four, at least five, or even at least six polymerizable functional groups.

For ease of reference in this specification and in the claims, the term “monomer” or “monomers” as used herein with regard to the structural materials that form the wall polymer of the delivery particles is to be understood as monomers, but also is inclusive of oligomers and/or prepolymers formed of the specific monomers.

As used herein the term “water soluble material” means a material that has a solubility of at least 0.5% wt in water at 60° C.

As used herein the term “oil soluble” means a material that has a solubility of at least 0.1% wt in the core of interest at 50° C.

As used herein the term “oil dispersible” means a material that can be dispersed at least 0.1% wt in the core of interest at 50° C. without visible agglomerates.

### SUMMARY OF THE INVENTION

The invention describes a delivery particle comprising a core material and a shell encapsulating the core material. The core material can comprise a benefit agent. The shell comprises a polymer. More particularly, the polymer comprises the reaction product of:
i) an isocyanate or acid chloride or oil soluble bi- or multi-functional (meth)acrylate with
ii) an amine-containing natural material having free amino moieties, and
iii) an α, β -unsaturated compound, the α, β -unsaturated compound forming C-N covalent bonds with the amine moieties of the natural material. The % wt ratio of the isocyanate to amine-containing natural material to α, β -unsaturated compound being in the ranges from 0.1:90:9.9 to 20:10:70 based on weight of the polymer.

The α, β -unsaturated compound forms C-N covalent bonds with the free amino groups of the natural polymer. The natural material can be selected from chitosan, chitin, gelatin, amine containing starch, amino sugar, polylysine, or hyaluronic acid. Without bound by theory, the C-N covalent bonds are formed via a conjugate nucleophilic addition reaction involving N-nucleophiles, such as the free amino moieties on the natural polymers and electron-deficient alkene molecules, such as α, β-unsaturated esters.

The α, β -unsaturated compound can be selected from water-soluble or dispersible acrylates, methacrylates, alkyl acrylates, α, β -unsaturated esters, acrylic acid, acrylamides, vinyl ketones, vinyl sulfones, vinyl phosphonates, acrylonitrile derivatives or mixtures thereof. For clarity the water soluble or dispersible acrylates generally will differ from the oil soluble oil soluble bi- or multi- functional acrylates. In certain instances, a similar material may be applied for each phase.

Water soluble or dispersible is an ability to dissolve or to be dispersed in water. Water soluble material generally will have a solubility in water of at least 0.01 g per 100 ml of water, or even more than 0.03 g per 100 ml of water at 25° C., but usually more than 1 g/100 cc. Water dispersible means that the material is dispersed at least 0.1 % wt without visible agglomerates.

In general, an oil soluble monomer is soluble or dispersible in the oil phase, typically soluble at least to the extent of 0.1 grams in 100 ml of the oil, or dispersible or emulsifiable therein at 50° C.

In embodiments, the α, β -unsaturated compound is a monofunctional, bifunctional, or multifunctional polymeric compound or mixtures thereof. The α, β -unsaturated compound can be selected to be anionic charged. Alternatively, the α, β -unsaturated compound can be cationic charged.

The delivery particle zeta potential of the delivery particle is from -100 mV - +200 mV at pH 3 and -200 mV - +100 mV at pH 10.

A portion of the free amino moieties of the natural material are reacted with the α, β -unsaturated compound via an Aza-Michael Addition reaction. Additionally, a portion of the free amino moieties of the natural material are reacted with an isocyanate, acid chloride, or (meth)acrylate to form a urea, amide, or an amino ester bond respectively.

In embodiments where a portion of the free amino moieties of the natural material is reacted with isocyanate, the isocyanate can be selected from the group consisting of a polyisocyanurate of toluene diisocyanate, a trimethylol propane adduct of toluene diisocyanate, a trimethylol propane adduct of xylylene diisocyanate, methylene diphenyl isocyanate, toluene diisocyanate, tetramethylxylidene diisocyanate, naphthalene-1,5-diisocyanate, and phenylene diisocyanate.

In embodiments where a portion of the free amino moieties of the natural material is reacted with acid chloride, the acid chloride can be selected from terephthaloyl chloride, isophthaloyl chloride, phthaloyl chloride, 1,3,5-benzenetricarbonyl trichloride, adipoyl chloride, glutaryl chloride, or sebacoyl chloride.

In embodiments where a portion of the free amino moieties of the natural material is reacted with oil soluble (meth)acrylate, The oil soluble (meth)acrylate is selected from group consisting of bi-functional (meth)acrylate, tri-functional (meth)acrylate, tetra-functional (meth)acrylate, penta-functional (meth)acrylate, hexa-functional (meth)acrylate, hepta-functional (meth)acrylate, and mixtures thereof. The oil soluble multifunctional (meth)acrylate can be a multifunctional acrylate or methacrylate monomer or oligomer or pre-polymer and can include di-; tri-; tetra-penta-; hexa-; hepta-; or octa-functional acrylate esters, methacrylate esters and multi-functional polyurethane acrylate esters.

The α, β -unsaturated water-soluble or dispersible acrylates can be selected from ester-based acrylate, ethylene glycol-based acrylate, propylene glycol-based acrylate, amino ester-based acrylate. Ester-based acrylate:

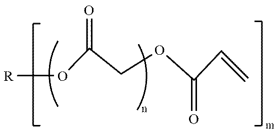

Ethylene glycol-based acrylate:

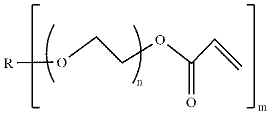

Propylene glycol-based acrylate:

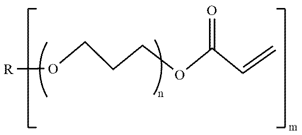

Amino ester-based acrylate:

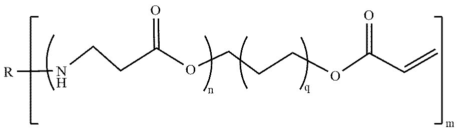

m=1-6; n=1-200; q=0-24 wherein R is as shown in Structure V

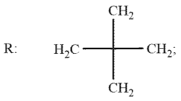

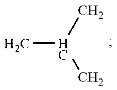

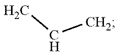

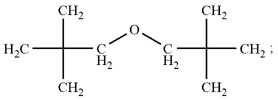

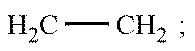

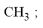

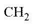

The α, β -unsaturated water-soluble or dispersible acrylates for illustration may include, but not by way of limitation, 2-carboxyethyl acrylate, 2-carboxyethyl acrylate oligomers, 2-carboxypropyl acrylate, 4-acryloyloxyphenylacetic acid, carboxyoctyl acrylate, tripropylene glycol diacrylate, ethoxylated bisphenol diacrylate, dipropylene glycol diacrylate, alkoxylated hexanediol diacrylate, alkoxylated cyclohexane dimethanol diacrylate, propoxylated neopentyl glycol diacrylate, trimethylolpropane triacrylate, pentaerythritol triacrylate, ethoxylated trimethylolpropane triacrylate, propoxylated trimethylolpropane triacrylate, propoxylated glyceryl triacrylate, ditrimethylolpropane tetraacrylate, dipentaerythritol pentaacrylate, ethoxylated pentaerythritol tetraacrylate, glycerol tri(meth)acrylate, ethylene glycol diacrylate, di-, tri-, tetra-, or pentaethylene glycol diacrylate, dipropylene glycol diacrylate, polyethylene glycol diacrylate, 2-ethylhexyl acrylate, 2-hydroxyethyl (meth)acrylate, cyanoethyl acrylate, 2-hydroxypropyl acrylate, lauryl acrylate, cyclohexyl acrylate, tetrahydrofurfuryl acrylate, chlorobenzyl acrylate, amino alkylacrylate, ethylaminoethyl (meth)acrylate, aminoethyl (meth)acrylate, tertiarybutyl aminoethyl (meth)acrylate, diethylamino (meth)acrylate, diethylaminoethyl (meth)acrylate, dimethylaminoethyl (meth)acrylate independently or a combination of the foregoing.

The oil-soluble or dispersible multifunctional (meth)acrylate monomers and oligomers contain two or more double bonds, preferably two or more acrylate or methacrylate functional groups. Suitable monomers and oligomers include, by way of illustration and not limitation, allyl methacrylate; triethylene glycol dimethacrylate; ethylene glycol dimethacrylate; diethylene glycol dimethacrylate; aliphatic or aromatic urethane acrylates, such as hexa-functional aromatic urethane acrylates; ethoxylated aliphatic difunctional urethane methacrylates; aliphatic or aromatic urethane methacrylates, such as tetra-functional aromatic methacrylates; epoxy acrylates; epoxymethacrylates; tetraethylene glycol dimethacrylate; polyethylene glycol dimethacrylate; 1,3 butanediol diacrylate; 1,4-butanediol dimethacrylate; 1,4-butanediol diacrylate; diethylene glycol diacrylate; 1,6 hexanediol diacrylate; 1,6 hexanediol dimethacrylate; neopentyl glycol diacrylate; polyethylene glycol diacrylate; tetraethylene glycol diacrylate, triethylene glycol diacrylate; 1,3 butylene glycol dimethacrylate; tripropylene glycol diacrylate, ethoxylated bisphenol A diacrylate; ethoxylated bisphenol A dimethylacrylate; dipropylene glycol diacrylate; alkoxylated hexanediol diacrylate; alkoxylated cyclohexane dimethanol diacrylate; propoxylated neopentyl glycol diacrylate, trimethylolpropane trimethacrylate; trimethylolpropane triacrylate; pentaerythritol triacrylate; pentaerythritol tetramethacrylate; ethoxylated trimethylolpropane triacrylate; propoxylated trimethylolpropane triacrylate; propoxylated glyceryl triacrylate; ditrimethylolpropane tetraacrylate; dipentaerythritol pentaacrylate; ethoxylated pentaerythritol tetraacrylate; bis-phenol A diacrylate; bis-phenol A dimethacrylate, hexa-functional aromatic urethane acrylate; hexa-functional aromatic urethane methacrylate; independently or a combination of the foregoing.

In embodiments, the benefit agent comprising the core is a fragrance, preferably a fragrance comprising perfume raw materials characterized by a logP of from about 2.5 to about 4.5. The core can comprise in addition a partitioning modifier selected from the group consisting of isopropyl myristate, vegetable oil, modified vegetable oil, mono-, di-, and tri-esters of C4-C24 fatty acids, dodecanophenone, lauryl laurate, methyl behenate, methyl laurate, methyl palmitate, methyl stearate, and mixtures thereof, preferably isopropyl myristate.

In certain embodiments, the wall has a biodegradability above 30% CO2 in 60 days following an OECD 301B test, preferably above 40% CO2, more preferably above 50% CO2, even more preferably above 60% CO2.

Optionally or alternatively, the wall of the delivery particles further comprises a coating material, preferably wherein the coating material is selected from the group consisting of poly(meth)acrylate, poly(ethylene-maleic anhydride), polyamine, wax, polyvinylpyrrolidone, polyvinylpyrrolidone co-polymers, polyvinylpyrrolidone-ethyl acrylate, polyvinylpyrrolidone-vinyl acrylate, polyvinylpyrrolidone methacrylate, polyvinylpyrrolidone/vinyl acetate, polyvinyl acetal, polyvinyl butyral, polysiloxane, poly(propylene maleic anhydride), maleic anhydride derivatives, co-polymers of maleic anhydride derivatives, polyvinyl alcohol, styrene-butadiene latex, gelatine, gum arabic, carboxymethyl cellulose, carboxymethyl hydroxyethyl cellulose, hydroxyethyl cellulose, other modified celluloses, sodium alginate, chitosan, chitin, casein, pectin, modified starch, polyvinyl acetal, polyvinyl butyral, polyvinyl methyl ether/maleic anhydride, polyvinyl pyrrolidone and its co polymers, poly(vinyl pyrrolidone/methacrylamidopropyl trimethyl ammonium chloride), polyvinylpyrrolidone/vinyl acetate, polyvinyl pyrrolidone/dimethylaminoethyl methacrylate, polyvinyl amines, polyvinyl formamides, polyallyl amines, copolymers of polyvinyl amines, and mixtures thereof.

The invention also describes a process of forming a population of delivery particles, the delivery particles comprising a core material and a shell encapsulating the core material, wherein the core material comprises a benefit agent; and, wherein the shell comprises a polymer, the polymer comprising the reaction product of:
i) an isocyanate or acid chloride or bi- or multi-functional (meth)acrylate with
ii) an amine-containing natural material having free amino moieties, and
iii) an α, β -unsaturated compound;
the process comprising:
i) forming a water phase comprising dissolving or dispersing in water an amine-containing natural material;
ii) forming an oil phase by mixing together a benefit agent, preferably perfume, optionally a partitioning modifier, and optionally a solvent, together with a shell-forming materials selected from the group consisting of an isocyanate, an acid chloride, and an oil-soluble bi- or multi- functional (meth)acrylate
iii) emulsifying the oil phase into the water phase to form an emulsion and heating the emulsion;
iv) adding to the emulsion an α, β -unsaturated compound comprising a water-soluble or dispersible acrylate, an alkyl acrylate, an α, β -unsaturated ester, an acrylic acid, an acrylamide, a vinyl ketone, a vinyl sulfone, a vinyl phosphonate, or an acrylonitrile derivative, and mixing together the emulsion and α, β -unsaturated compound
v) the α, β -unsaturated compound forming C-N covalent bonds with a portion of the amine groups of the natural material.

The α, β -unsaturated compounds undergo conjugate addition with nucleophiles, namely, the free amine groups of the amine-containing natural material. The α, β -unsaturated compounds are electron deficient at the unsaturated bonds. This conjugate addition of nucleophiles to the electron deficient unsaturated sites results in formation of C-N covalent bonds with a portion of the amine groups of the natural material.

By mixing, microwave promoting, or heating, the free amines of the amine-containing natural material react as nucleophiles covalently bonding at the unsaturated site of the α, β -unsaturated compound.

The delivery particle has a leakage of below about 50%, as determined by the Leakage Test described in the TEST METHODS Section.

In further constructs, the delivery particles of the invention can be fashioned into new articles by incorporation into various articles of manufacture. Such article can be selected from the group consisting of an agricultural formulation, a slurry encapsulating an agricultural active, a population of dry microcapsules encapsulating an agricultural active, an agricultural formulation encapsulating an insecticide, and an agricultural formulation for delivering a preemergent herbicide. The agricultural active can be selected from the group consisting of an agricultural herbicide, an agricultural pheromone, an agricultural pesticide, an agricultural nutrient, an insect control agent and a plant stimulant.

### BRIEF DESCRIPTION OF THE DRAWINGS

FIG. 1 illustrates the measured zeta potential of encapsulates according to the invention.

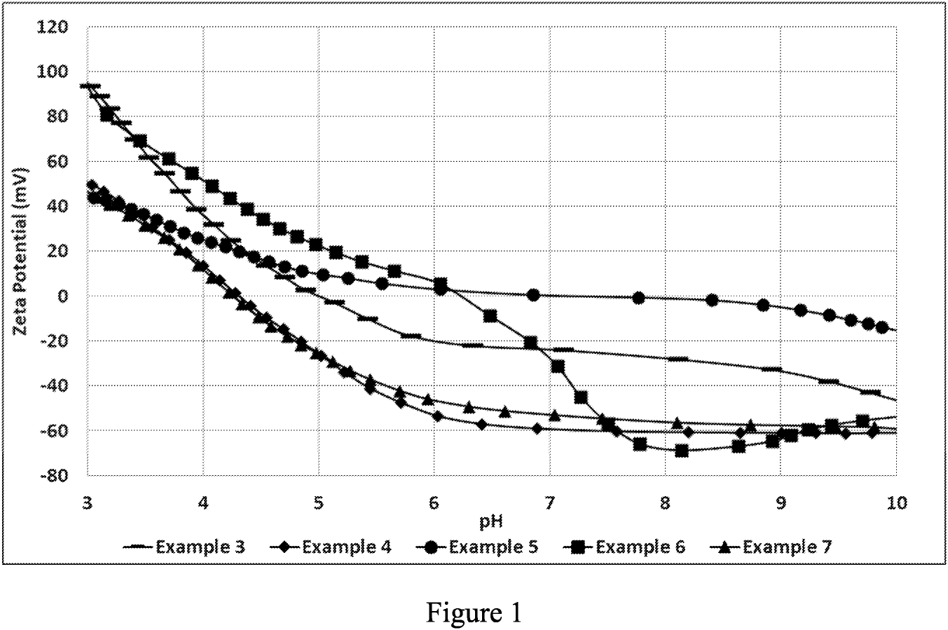

### DETAILED DESCRIPTION

The invention describes a delivery particle comprising a core material and a shell encapsulating the core material. The core material can comprise a benefit agent. The shell comprises a polymer. More particularly, the polymer comprises the reaction product of:
i) an isocyanate or acid chloride or acrylate with
ii) an amine-containing natural material having free amino moieties, and
iii) an α, β -unsaturated compound, the α, β -unsaturated compound forming C-N covalent bonds with the amine moieties of the natural material. The % wt ratio of the isocyanate to amine-containing natural material to α, β -unsaturated compound being in the ranges from 0.1:90:9.9 to 20:10:70 based on weight of the polymer.

The α, β -unsaturated compound forms C-N covalent bonds with the free amino groups of the natural polymer. The natural material is selected from chitosan, chitin, gelatin, amine containing starch, amino sugar, polylysine, or hyaluronic acid.

The α, β -unsaturated compound can be selected, by way of illustration and not limitation, from water-soluble or dispersible acrylates, methacrylates, alkyl acrylates, α, β -unsaturated esters, acrylic acid, acrylamides, vinyl ketones, vinyl sulfones, vinyl phosphonates, acrylonitrile derivatives or mixtures thereof. Specific example of α, β -unsaturated compounds useful in the invention include α, β -unsaturated esters including: α, β -unsaturated carboxylic acid esters and acrylic or methacrylic esters. Exemplary acrylamides include: acrylamide, methacrylamide, n-isopropyl acrylamide, (3-acrylamidopropyl) trimethylammonium chloride, 2-acrylamido-2-methyl-1-propanesulfonic acid. Exemplary vinyl ketones include: vinyl methyl ketone, vinyl ethyl ketone, vinyl hexyl ketone, vinyl isopropenyl ketone, vinyl isopropyl ketone, α, β -unsaturated compounds can include vinyl sulphones, vinyl phosphonates and acrylonitrile derivatives.

To create the delivery particle of the invention a water phase is prepared, comprising a water solution or dispersion of an amine-containing natural material having free amino moieties. The amine containing natural material is a bio-based material. Such materials for example include chitosan. The amine-containing natural material is dispersed in water. In the case of chitosan, the material is hydrolyzed thereby protonating at least a portion of the amine groups and facilitating dissolving in water. Hydrolysis is carried out with heating for a period at an acidic pH such as about 5 or 5.5.

The hydrolyzed amine-containing natural material solution is then used for a first reaction with the isocyanate or acid chloride or oil-soluble bi- or multi- functional (meth)acrylate. This is accomplished by preparing an oil phase containing the core material comprising a benefit agent and the shell-forming isocyanate or acid chloride or oil-soluble bi- or multi- functional (meth)acrylate. An emulsion is formed when the oil phase is combined with the water phase under high shear agitation. The emulsion is heated such as to approximately 60 to 95° C., or even 60 to 80° C., or even to 70 to 80° C. initiating reaction with oil phase isocyanate or acid chloride or oil-soluble bi- or multi- functional (meth)acrylate. As reaction proceeds, a second cross-linker comprising an α, β -unsaturated compound is added to the emulsion. The α, β -unsaturated compound forms C-N covalent bonds with the amine moieties of the natural material. The α, β -unsaturated compound is added as the first emulsion forms, or added during emulsification, but while a portion of amines remain available for linking with the added α, β -unsaturated compound.

The α, β -unsaturated compound is selected from water-soluble or dispersible materials, such as a second acrylate. The water soluble or dispersible materials can be acrylate, alkyl acrylate, or an α, β -unsaturated ester, or an acrylic acid, an acrylamide, a vinyl ketone, a vinyl sulfone, a vinyl phosphonate, an acrylonitrile derivative or mixtures thereof. The α, β -unsaturated compound comprises further shell forming material, namely the shell forming material from the water phase and is a second crosslinker.

The invention can be illustrated, such as with gelatin as the natural material. In an embodiment, to create the delivery particle of the invention, a water phase is prepared comprising a water solution or dispersion of an amine-containing natural material having free amino moieties. The amine containing natural material is selected to be a bio-based material. Such material for example can comprise gelatin, such as type B Bovine gelatin. The amine-containing natural material is dispersed in water with heating at 50° C. After dissolution the solution is cooled to about 25° C. An oil phase is prepared with a perfume and an optional partitioning modifier such as isopropyl myristate, together with an isocyanate or acid chloride or oil-soluble bi- or multifunctional (meth)acrylate. The oil phase is added to the water phase under high shear milling to form an emulsion. A water-soluble or dispersible acrylate, an alkyl acrylate, an α, β -unsaturated ester, an acrylic acid, an acrylamide, a vinyl ketone, a vinyl sulfone, a vinyl phosphonate, an acrylonitrile derivative or mixtures of the foregoing are added. For example, the water soluble or dispersible α, β - unsaturated compound can be trimetholpropane triacrylate as illustrated in specific examples herein.

The gelatin reacts with the isocyanate or acid chloride or oil-soluble bi- or multi-functional (meth)acrylate. This is accomplished by preparing an oil phase containing the core material comprising a benefit agent and the shell-forming isocyanate or acid chloride or oil-soluble bi- or multi- functional (meth)acrylate. An emulsion is formed when the oil phase is combined with the water phase under high shear agitation. The emulsion is heated such as to approximately 60 to 95° C., or even 60 to 80 °C, or even to 70 to 80° C., initiating reaction with the oil phase isocyanate or acid chloride or oil-soluble bi- or multi- functional (meth)acrylate. As reaction proceeds, the second cross-linker comprising the α, β -unsaturated compound is added to the emulsion. The α, β -unsaturated compound forms C-N covalent bonds with the amine moieties of the gelatin. The α, β -unsaturated compound is added as the first emulsion forms, or added during emulsification, but while a portion of amines remain available for linking with the added α, β -unsaturated compound.

The α, β -unsaturated compound is selected from water-soluble or dispersible materials, such as acrylate, alkyl acrylate, or an α, β -unsaturated ester, or an acrylic acid, an acrylamide, a vinyl ketone, a vinyl sulfone, a vinyl phosphonate, an acrylonitrile derivative or mixtures thereof. The α, β -unsaturated compound comprises further shell forming material, namely the shell forming material from the water phase and is a second cross-linker.

The oil phase is prepared by dissolving an isocyanate (or alternatively acid chloride or multifunctional (meth)acrylate) such as trimers of xylylene diisocyanate (XDI) or polymers of methylene diphenyl isocyanate (MDI), in oil at 25° C. Diluents, for example isopropyl myristate, may be used to adjust the hydrophilicity of the oil phase. The oil phase is then added into the water phase and milled at high speed to obtain a targeted size. The emulsion is then cured in one or more heating steps, such as heating to 40° C. in 30 minutes and holding at 40° C. for 60 minutes. Times and temperatures are approximate. The temperature and time are selected to be sufficient to form and cure a shell at the interface of the droplets of the oil phase with the water continuous phase. For example, the emulsion is heated to 85° C. in 60 minutes and then held at 85° C. for 360 minutes to cure the capsules. The slurry is then cooled to room temperature.

Volume weighted median particle size of delivery particles according to the invention can range from 5 microns to 150 microns, or even from 10 to 50 microns, preferably 15 to 50 microns.

The isocyanates useful in the invention are to be understood for purposes hereof as isocyanate monomer, isocyanate oligomer, isocyanate prepolymer, or dimer or trimer of an aliphatic or aromatic isocyanate. All such monomers, prepolymers, oligomers, or dimers or trimers of aliphatic or aromatic isocyanates are intended encompassed by the term “isocyanate” as used herein.

The isocyanate is an aliphatic or aromatic monomer, oligomer or prepolymer, usefully of two or more isocyanate functional groups. The isocyanate, for example, can be selected from aromatic toluene diisocyanate and its derivatives used in wall formation for encapsulates, or aliphatic monomer, oligomer or prepolymer, for example, hexamethylene diisocyanate and dimers or trimers thereof, or 3,3,5-trimethyl-5-isocyanatomethyl-1-isocyanato cyclohexane tetramethylene diisocyanate. The polyisocyanate can be selected from 1,3-diisocyanato-2-methylbenzene, hydrogenated MDI, bis(4-isocyanatocyclohexyl) methane, dicyclohexylmethane-4,4’-diisocyanate, and oligomers and prepolymers thereof. This listing is illustrative and not intended to be limiting of the polyisocyanates useful in the invention.

The isocyanates useful in the invention comprise isocyanate monomers, oligomers or prepolymers, or dimers or trimers thereof, having at least two isocyanate groups. Optimal cross-linking can be achieved with isocyanates having at least three functional groups.

Isocyanates, for purposes of the invention, are understood as encompassing any isocyanate monomer, oligomer, prepolymer or polymer having at least two isocyanate groups and comprising an aliphatic or aromatic moiety in the monomer, oligomer or prepolymer. If aromatic, the aromatic moiety can comprise a phenyl, a toluyl, a xylyl, a naphthyl or a diphenyl moiety, more preferably a toluyl or a xylyl moiety. Aromatic polyisocyanates, for purposes hereof, can include diisocyanate derivatives such as biurets and polyisocyanurates. The polyisocyanate, when aromatic, can be, but is not limited to, methylene diphenyl isocyanate, toluene diisocyanate, tetramethylxylidene diisocyanate, polyisocyanurate of toluene diisocyanate (commercially available from Bayer under the tradename Desmodur® RC), trimethylol propane-adduct of toluene diisocyanate (commercially available from Bayer under the tradename Desmodur® L75), or trimethylol propane-adduct of xylylene diisocyanate (commercially available from Mitsui Chemicals under the tradename Takenate® D-110N), naphthalene-1,5-diisocyanate, and phenylene diisocyanate.

Isocyanate, which is aliphatic, is understood as a monomer, oligomer, prepolymer or polymer polyisocyanate which does not comprise any aromatic moiety. There is a preference for aromatic polyisocyanate, however, aliphatic polyisocyanates and blends thereof are useful. Aliphatic polyisocyanates include a trimer of hexamethylene diisocyanate, a trimer of isophorone diisocyanate, a trimethylol propane-adduct of hexamethylene diisocyanate (available from Mitsui Chemicals) or a biuret of hexamethylene diisocyanate (commercially available from Bayer under the tradename Desmodur® N 100).

The capsule shell could also be reinforced using additional co-crosslinkers such as multifunctional amines and/or polyamines such as diethylene triamine (DETA), polyethylene imine, and polyvinyl amine. Core

The microcapsules of the present teaching include a benefit agent which comprises one or more ingredients that are intended to be encapsulated. The benefit agent is selected from a number of different materials such as chromogens and dyes, flavorants, perfumes, sweeteners, fragrances, oils, fats, pigments, cleaning oils, pharmaceuticals, pharmaceutical oils, perfume oils, mold inhibitors, antimicrobial agents, fungicides, bactericides, disinfectants, adhesives, phase change materials, scents, fertilizers, nutrients, and herbicides: by way of illustration and without limitation. The benefit agent and oil comprise the core. The core can be a liquid or a solid. With cores that are solid at ambient temperatures, the wall material can usefully enwrap less than the entire core for certain applications where availability of, for example, an agglomerate core is desired on application. Such uses can include scent release, cleaning compositions, emollients, cosmetic delivery and the like. Where the microcapsule core is phase change material, uses can include such encapsulated materials in mattresses, pillows, bedding, textiles, sporting equipment, medical devices, building products, construction products, HVAC, renewable energy, clothing, athletic surfaces, electronics, automotive, aviation, shoes, beauty care, laundry, and solar energy.

The core constitutes the material encapsulated by the microcapsules. Typically, particularly when the core material is a liquid material, the core material is combined with one or more of the compositions from which the internal wall of the microcapsule is formed or solvent for the benefit agent or partitioning modifier. If the core material can function as the oil solvent in the capsules, e.g., acts as the solvent or carrier for either the wall forming materials or benefit agent, it is possible to make the core material the major material encapsulated, or if the carrier itself is the benefit agent, can be the total material encapsulated. Usually however, the benefit agent is from 0.01 to 99 weight percent of the capsule internal contents, preferably 0.01 to about 65 by weight of the capsule internal contents, and more preferably from 0.1 to about 45 % by weight of the capsule internal contents. With certain applications, the core material can be effective even at just trace quantities.

Where the benefit agent is not itself sufficient to serve as the oil phase or solvent, particularly for the wall forming materials, the oil phase can comprise a suitable carrier and/or solvent. In this sense, the oil is optional, as the benefit agent itself can at times be the oil. These carriers or solvents are generally an oil, preferably have a boiling point greater than about 80° C. and low volatility and are non-flammable. Though not limited thereto, they preferably comprise one or more esters, preferably with chain lengths of up to 18 carbon atoms or even up to 42 carbon atoms and/or triglycerides such as the esters of C6 to C12 fatty acids and glycerol. Exemplary carriers and solvents include, but are not limited to: ethyldiphenylmethane; isopropyl diphenylethane; butyl biphenyl ethane; benzylxylene; alkyl biphenyls such as propylbiphenyl and butylbiphenyl; dialkyl phthalates e.g. dibutyl phthalate, dioctylphthalate, dinonyl phthalate and ditridecylphthalate; 2,2,4-trimethyl-1,3-pentanediol diisobutyrate; alkyl benzenes such as dodecyl benzene; alkyl or aralkyl benzoates such as benzyl benzoate; diaryl ethers; di(aralkyl)ethers and aryl aralkyl ethers; ethers such as diphenyl ether, dibenzyl ether and phenyl benzyl ether; liquid higher alkyl ketones (having at least 9 carbon atoms); alkyl or aralkyl benzoates, e.g., benzyl benzoate; alkylated naphthalenes such as dipropylnaphthalene; partially hydrogenated terphenyls; high-boiling straight or branched chain hydrocarbons; alkaryl hydrocarbons such as toluene; vegetable and other crop oils such as canola oil, soybean oil, corn oil, sunflower oil, cottonseed oil, lemon oil, olive oil and pine oil; methyl esters of fatty acids derived from transesterification of vegetable and other crop oils, methyl ester of oleic acid, esters of vegetable oil, e.g. soybean methyl ester, straight chain paraffinic aliphatic hydrocarbons, and mixtures of the foregoing.

Useful benefit agents include perfume raw materials, such as alcohols, ketones, aldehydes, esters, ethers, nitriles, alkenes, fragrances, fragrance solubilizers, essential oils, phase change materials, lubricants, colorants, cooling agents, preservatives, antimicrobial or antifungal actives, herbicides, antiviral actives, antiseptic actives, antioxidants, biological actives, deodorants, emollients, humectants, exfoliants, ultraviolet absorbing agents, self-healing compositions, corrosion inhibitors, sunscreens, silicone oils, waxes, hydrocarbons, higher fatty acids, essential oils, lipids, skin coolants, vitamins, sunscreens, antioxidants, glycerine, catalysts, bleach particles, silicon dioxide particles, malodor reducing agents, dyes, brighteners, antibacterial actives, antiperspirant actives, cationic polymers and mixtures thereof. Phase change materials useful as benefit agents can include, by way of illustration and not limitation, paraffinic hydrocarbons having 13 to 28 carbon atoms, various hydrocarbons such n-octacosane, n-heptacosane, n-hexacosane, n-pentacosane, n-tetracosane, n-tricosane, n-docosane, n-heneicosane, n-eicosane, n-nonadecane, octadecane, n-heptadecane, n-hexadecane, n-pentadecane, n-tetradecane, n-tridecane. Phase change materials can alternatively, optionally in addition include crystalline materials such as 2,2-dimethyl-1,3-propanediol, 2-hydroxymethyl-2-methyl-1, 3-propanediol, acids of straight or branched chain hydrocarbons such as eicosanoic acid and esters such as methyl palmitate, fatty alcohols and mixtures thereof.

Preferably, in the case of fragrances, a perfume oil acts as benefit agent and solvent for the wall forming material, as illustrated in the examples herein.

Optionally the water phase may include an emulsifier. Non-limiting examples of emulsifiers include water-soluble salts of alkyl sulfates, alkyl ether sulfates, alkyl isothionates, alkyl carboxylates, alkyl sulfosuccinates, alkyl succinamates, alkyl sulfate salts such as sodium dodecyl sulfate, alkyl sarcosinates, alkyl derivatives of protein hydrolyzates, acyl aspartates, alkyl or alkyl ether or alkylaryl ether phosphate esters, sodium dodecyl sulphate, phospholipids or lecithin, or soaps, sodium, potassium or ammonium stearate, oleate or palmitate, alkylarylsulfonic acid salts such as sodium dodecylbenzenesulfonate, sodium dialkylsulfosuccinates, dioctyl sulfosuccinate, sodium dilaurylsulfosuccinate, poly(styrene sulfonate) sodium salt, isobutylene-maleic anhydride copolymer, gum arabic, sodium alginate, carboxymethylcellulose, cellulose sulfate and pectin, poly(styrene sulfonate), isobutylene-maleic anhydride copolymer, carrageenan, sodium alginate, pectic acid, tragacanth gum, almond gum and agar; semi-synthetic polymers such as carboxymethyl cellulose, sulfated cellulose, sulfated methylcellulose, carboxymethyl starch, phosphated starch, lignin sulfonic acid; and synthetic polymers such as maleic anhydride copolymers (including hydrolyzates thereof), polyacrylic acid, polymethacrylic acid, acrylic acid butyl acrylate copolymer or crotonic acid homopolymers and copolymers, vinyl benzenesulfonic acid or 2-acrylamido-2-methylpropanesulfonic acid homopolymers and copolymers, and partial amide or partial ester of such polymers and copolymers, carboxy modified polyvinyl alcohol, sulfonic acid-modified polyvinyl alcohol and phosphoric acid-modified polyvinyl alcohol, phosphated or sulfated tristyrylphenol ethoxylates, palmitamidopropyltrimonium chloride (Varisoft PATC™, available from Degussa Evonik, Essen, Germany), distearyl dimonium chloride, cetyltrimethylammonium chloride, quaternary ammonium compounds, fatty amines, aliphatic ammonium halides, alkyldimethylbenzylammonium halides, alkyldimethylethylammonium halides, polyethyleneimine, poly(2-dimethylamino)ethyl methacrylate) methyl chloride quaternary salt, poly(1-vinylpyrrolidone-co-2-dimethylaminoethyl methacrylate), poly(acrylamide-co-diallyldimethylammonium chloride), poly(allylamine), poly[bis(2-chloroethyl) ether-alt-1,3-bis[3-(dimethylamino)propyl]urea] quaternized, and poly(dimethylamine-co-epichlorohydrin-co-ethylenediamine), condensation products of aliphatic amines with alkylene oxide, quaternary ammonium compounds with a long-chain aliphatic radical, e.g. distearyldiammonium chloride, and fatty amines, alkyldimethylbenzylammonium halides, alkyldimethylethylammonium halides, polyalkylene glycol ether, condensation products of alkyl phenols, aliphatic alcohols, or fatty acids with alkylene oxide, ethoxylated alkyl phenols, ethoxylated aryl phenols, ethoxylated polyaryl phenols, carboxylic esters solubilized with a polyol, polyvinyl alcohol, polyvinyl acetate, or copolymers of polyvinyl alcohol polyvinyl acetate, polyacrylamide, poly(N-isopropylacrylamide), poly(2-hydroxypropyl methacrylate), poly(-ethyl-2-oxazoline), poly(2-isopropenyl-2-oxazoline-co-methyl methacrylate), poly(methyl vinyl ether), and polyvinyl alcohol-co-ethylene), and cocoamidopropyl betaine. Emulsifier, if employed, is typically from about 0.1 to 40% by weight, preferably 0.2 to about 15% by weight, more typically 0.5 to 10% be weight, based on total weight of the formulation

The microcapsules may encapsulate a partitioning modifier in addition to the benefit agent. Non-limiting examples of partitioning modifiers include isopropyl myristate, mono-, di-, and tri-esters of C₄-C₂₄ fatty acids, castor oil, mineral oil, soybean oil, hexadecanoic acid, methyl ester isododecane, isoparaffin oil, polydimethylsiloxane, brominated vegetable oil, and combinations thereof. Microcapsules may also have varying ratios of the partitioning modifier to the benefit agent so as to make different populations of microcapsules that may have different bloom patterns. Such populations may also incorporate different perfume oils so as to make populations of microcapsules that display different bloom patterns and different scent experiences. US 2011-0268802 discloses other non-limiting examples of microcapsules and partitioning modifiers and is hereby incorporated by reference.

Optionally, if desired, the delivery particles can be dewatered such as through decanting, filtration, centrifuging or other separation technique. Alternatively, the aqueous slurry delivery particles can be spray dried.

In some examples of the process and compositions, the microcapsules may consist of one or more distinct populations. The composition may have at least two different populations of microcapsules that vary in the exact make-up of the perfume oil and in the median particle size and/or partitioning modifier to perfume oil (PM:PO) weight ratio. In some examples, the composition includes more than two distinct populations that vary in the exact make up the perfume oil and in their fracture strengths. In some further examples, the populations of microcapsules can vary with respect to the weight ratio of the partitioning modifier to the perfume oil(s). In some examples, the composition can include a first population of microcapsules having a first ratio that is a weight ratio of from 2:3 to 3:2 of the partitioning modifier to a first perfume oil and a second population of microcapsules having a second ratio that is a weight ratio of less than 2:3 but greater than 0 of the partitioning modifier to a second perfume oil.

In some embodiments, each distinct population of microcapsules is preparable in a distinct slurry. For example, the first population of microcapsules can be contained in a first slurry and the second population of microcapsules contained in a second slurry. It is to be appreciated that the number of distinct slurries for combination is without limit and a choice of the formulator such that 3, 10, or 15 distinct slurries may be combined. The first and second populations of microcapsules may vary in the exact make up the perfume oil and in the median particle size and/or PM:PO weight ratio.

In some embodiments, the composition, can be prepared by combining the first and second slurries with at least one adjunct ingredient and optionally packaged in a container. In some examples, the first and second populations of microcapsules can be prepared in distinct slurries and then spray dried to form a particulate. The distinct slurries may be combined before spray drying, or spray dried individually and then combined together when in particulate powder form. Once in powder form, the first and second populations of microcapsules may be combined with an adjunct ingredient to form the composition useful as a feedstock for manufacture of consumer, industrial, medical or other goods. In some examples, at least one population of microcapsules is spray dried and combined with a slurry of a second population of microcapsules. In some examples, at least one population of microcapsules is dried, prepared by spray drying, fluid bed drying, tray drying, or other such drying processes that are available.

In some examples, the slurry or dry particulates can include one or more adjunct materials such as processing aids selected from the group consisting of a carrier, an aggregate inhibiting material, a deposition aid, a particle suspending polymer, and mixtures thereof. Non-limiting examples of aggregate inhibiting materials include salts that can have a charge-shielding effect around the particle, such as magnesium chloride, calcium chloride, magnesium bromide, magnesium sulfate, and mixtures thereof. Non-limiting examples of particle suspending polymers include polymers such as xanthan gum, carrageenan gum, guar gum, shellac, alginates, chitosan; cellulosic materials such as carboxymethyl cellulose, hydroxypropyl methyl cellulose, cationically charged cellulosic materials; polyacrylic acid; polyvinyl alcohol; hydrogenated castor oil; ethylene glycol distearate; and mixtures thereof.

In some embodiments, the slurry can include one or more processing aids, selected from the group consisting of water, aggregate inhibiting materials such as divalent salts; particle suspending polymers such as xanthan gum, guar gum, carboxy methyl cellulose.

In other examples of the invention, the slurry can include one or more carriers selected from the group consisting of polar solvents, including but not limited to, water, ethylene glycol, propylene glycol, polyethylene glycol, glycerol; nonpolar solvents, including but not limited to, mineral oil, perfume raw materials, silicone oils, hydrocarbon paraffin oils, and mixtures thereof.

In some examples, said slurry may include a deposition aid that may comprise a polymer selected from the group comprising: polysaccharides, in one aspect, cationically modified starch and/or cationically modified guar; polysiloxanes; poly diallyl dimethyl ammonium halides; copolymers of poly diallyl dimethyl ammonium chloride and polyvinyl pyrrolidone; a composition comprising polyethylene glycol and polyvinyl pyrrolidone; acrylamides; imidazoles; imidazolinium halides; polyvinyl amine; copolymers of poly vinyl amine and N-vinyl formamide; polyvinyl formamide, polyvinyl alcohol; polyvinyl alcohol crosslinked with boric acid; polyacrylic acid; polyglycerol ether silicone cross-polymers; polyacrylic acids, polyacrylates, copolymers of polyvinylamine and polvyinylalcohol oligomers of amines, in one aspect a diethylenetriamine, ethylene diamine, bis(3-aminopropyl)piperazine, N,N-Bis-(3-aminopropyl)methylamine, tris(2-aminoethyl)amine and mixtures thereof; polyethyleneimine, a derivatized polyethyleneimine, in one aspect an ethoxylated polyethyleneimine; a polymeric compound comprising, at least two moieties selected from the moieties consisting of a carboxylic acid moiety, an amine moiety, a hydroxyl moiety, and a nitrile moiety on a backbone of polybutadiene, polyisoprene, polybutadiene/styrene, polybutadiene/acrylonitrile, carboxyl-terminated polybutadiene/acrylonitrile or combinations thereof; pre-formed coacervates of anionic surfactants combined with cationic polymers; polyamines and mixtures thereof.

In some additional examples to illustrate the invention, at least one population of microcapsules can be contained in an agglomerate and then combined with a distinct population of microcapsules and at least one adjunct material. Said agglomerate may comprise materials selected from the group consisting of silicas, citric acid, sodium carbonate, sodium sulfate, sodium chloride, and binders such as sodium silicates, modified celluloses, polyethylene glycols, polyacrylates, polyacrylic acids, zeolites and mixtures thereof.

Suitable equipment for use in the processes disclosed herein may include continuous stirred tank reactors, homogenizers, turbine agitators, recirculating pumps, paddle mixers, plough shear mixers, ribbon blenders, vertical axis granulators and drum mixers, both in batch and, where available, in continuous process configurations, spray dryers, and extruders. Such equipment can be obtained from Lodige GmbH (Paderborn, Germany), Littleford Day, Inc. (Florence, Ky., U.S.A.), Forberg AS (Larvik, Norway), Glatt Ingenieurtechnik GmbH (Weimar, Germany), Niro (Soeborg, Denmark), Hosokawa Bepex Corp. (Minneapolis, Minn., U.S.A.), Arde Barinco (New Jersey, U.S.A.).

### TEST METHODS

### Procedure for Determination of % Degradation

% degradation is determined by the “OECD Guideline for Testing of Chemicals” 301B CO₂ Evolution (Modified Sturm Test), adopted 17 Jul. 1992. For ease of reference, this test method is referred to herein as test method OECD 301B

### Procedure for Determination of Free Oil

This method measures the amount of oil in the water phase and uses as an internal standard solution 1 mg/ml dibutyl phthalate (DBP)/hexane.

Weigh a little more than 250 mgs of DBP into a small beaker and transfer to a 250 ml volumetric rinsing the beaker thoroughly. Fill with hexane to 250 ml.

Sample Prep: Weigh approximately 1.5-2 grams (40 drops) of the capsule slurry into a 20 ml scintillation vial and add 10 ml’s of the ISTD solution, cap tightly. Shaking vigorously several times over 30 minutes, pipette solution into an autosampler vial and analyze by GC.

Additional details. Instrumentation: HP5890 GC connected to HP Chem Station Software; Column: 5 m x 0.32 mm id with 1 µm DB-1 liquid phase; Temperature 50° C.; for 1 minute then heat to 320° C.; @ 15 deg/min; Injector: 275° C.; Detector: 325° C.; 2 ul injection.

Calculation: Add total peak area minus the area for the DBP for both the sample and calibration.
i) Calculate mg of free core oil:
$$\begin{matrix}\left(\frac{\text{Total area from sample}}{\text{Total area from calibration}}\right) \times \\ \text{mg of oil in calibration solution = mg of free oil}\end{matrix}$$
ii) Calculate % free core oil
$$\left(\frac{\text{mg of free core oil}}{\text{Sample wt} \text{.} \left(\text{mg}\right)}\right) \times 100 = \text{\% free core oil in wet slurry}$$

### Procedure for Determination of Benefit Agent Leakage

Obtain 2, one-gram samples of benefit agent particle composition. Add 1 gram (Sample 1) of particle composition to 99 grams of product matrix in which the particle will be employed. Age the particle containing product matrix (Sample 1) for 2 weeks at 35° C. in a sealed glass jar. The other one-gram sample (Sample 2) is similarly aged.

After 2 weeks, use filtration to recover the particle composition’s particles from the product matrix (Sample 1) and from the particle composition (Sample 2). Treat each particle sample with a solvent that will extract all the benefit agent from each samples’ particles. Inject the benefit agent containing solvent from each sample into a Gas Chromatograph and integrate the peak areas to determine the total quantity of benefit agent extracted from each sample.

Determine the percentage of benefit agent leakage by calculating the difference in the values obtained for the total quantity of benefit agent extracted from Sample 2 minus Sample 1, expressed as a percentage of the total quantity of benefit agent extracted from Sample 2, as represented in the equation below:

$$\begin{matrix}\text{Percentage of Benefit Agent Leakage} = \\ \left(\frac{S a m p l e 2 - S a m p l e 1}{S a m p l e 2}\right) \times 100\end{matrix}$$

Delivery particles can be prepared that exhibit positive zeta potentials. Such capsules have improved deposition efficiency, such as on fabrics.

### Sample Preparation for Biodegradability Measurements

The water soluble or water dispersible material is purified via crystallization till a purity of above 95% is achieved and dried before biodegradability measurement.

The oily medium comprising the benefit agent needs to be extracted from the delivery particle slurry in order to only analyze the polymer wall. Therefore, the delivery particle slurry is freeze dried to obtain a powder. Then, it is further washed with organic solvents via Soxhlet extraction method to extract the oily medium comprising the benefit agent till weight percentage of oily medium is below 5% based on total delivery particle polymer wall. Finally, the polymer wall is dried and analyzed.

Weight ratio of delivery particle to solvent is 1:3. Residual oily medium is determined by thermogravimetric analysis (60 minutes isotherm at 100° C. and another 60 minutes isotherm at 250° C.). The weight loss determined needs to be below 5%.

### OECD 301 B — Biodegradability Method

Accumulative CO₂ release is measured over 60 days following the guidelines of the Organisation for Economic Cooperation and Development (OECD) - OECD (1992), Test No. 301: Ready Biodegradability, OECD Guidelines for the Testing of Chemicals, Section 3, OECD Publishing, Paris, https://doi.org/10.1787/9789264070349-en.

### Leakage

The amount of benefit agent leakage from the benefit agent containing delivery particles is determined according to the following method:
i) Obtain two 1 g samples of the raw material slurry of benefit agent containing delivery particles.
ii) Add 1 g of the raw material slurry of benefit agent containing delivery particles to 99 g of the consumer product matrix in which the particles will be employed and label the mixture as Sample 1. Immediately use the second 1 g sample of raw material particle slurry in Step d below, in its neat form without contacting consumer product matrix, and label it as Sample 2.
iii) Age the delivery particle-containing product matrix (Sample 1) for 1 week at 35° C. in a sealed glass jar.
iv) Using filtration, recover the particles from both samples. The particles in Sample 1 (in consumer product matrix) are recovered after the aging step. The particles in Sample 2 (neat raw material slurry) are recovered at the same time that the aging step began for sample 1.
v) Treat the recovered particles with a solvent to extract the benefit agent materials from the particles.
vi) Analyze the solvent containing the extracted benefit agent from each sample, via chromatography.
vii) Integrate the resultant benefit agent peak areas under the curve and sum these areas to determine the total quantity of benefit agent extracted from each sample.
viii) Determine the percentage of benefit agent leakage by calculating the difference in the values obtained for the total quantity of benefit agent extracted from Sample 2 (S2) minus Sample 1 (S1), expressed as a percentage of the total quantity of benefit agent extracted from Sample 2 (s2), as represented in the equation below:
$$\% L e a k a g e = \left(\frac{S 2 - S 1}{S 2}\right) \times 10$$

### Volume Weighted Mean Particle Size

Particle size is measured using static light scattering devices, such as an Accusizer 780A, made by Particle Sizing Systems, Santa Barbara Calif. The instrument is calibrated from 0 to 300 µ using Duke particle size standards. Samples for particle size evaluation are prepared by diluting about 1 g emulsion, if the volume weighted mean particle size of the emulsion is to be determined, or 1 g of benefit agent containing delivery particles slurry, if the finished particles volume weighted mean particle size is to be determined, in about 5 g of de-ionized water and further diluting about 1 g of this solution in about 25 g of water.

About 1 g of the most dilute sample is added to the Accusizer and the testing initiated, using the autodilution feature. The Accusizer should be reading in excess of 9200 counts/second. If the counts are less than 9200 additional sample should be added. The Accusizer will dilute the test sample until 9200 counts/second and initiate the evaluation. After 2 minutes of testing the Accusizer will display the results, including volume-weighted mean size.

The broadness index can be calculated by determining the particle size at which 95% of the cumulative particle volume is exceeded (95% size), the particle size at which 5% of the cumulative particle volume is exceeded (5% size), and the median particle size (50% size-50% of the particle volume both above and below this size). Broadness Index = ((95% size) -(5% size)/50% size).

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

In the following examples, the abbreviations correspond to the materials listed in Table 1.

Table 1

| | | |
|---|---|---|
| Trade Name | Company/City | Material |
| Selvol 540 | Sekisui Specialty Chemicals, Dallas, TX | Polyvinyl alcohol |
| ChitoClear | Primex EHF, Siglufjordur, Iceland | chitosan |
| Takenate D-110N | Mitsui Chemicals America, Inc., Rye Brook, NY | aliphatic polyisocyanate prepolymer |
| CD9055 | Sartomer King of Prussia, PA | Acidic acrylate adhesion promoter |
| SR368 | Sartomer King of Prussia, PA | isocyanurate triacrylate |
| CN975 | Sartomer King of Prussia, PA | urethane acrylate oligomer |
| Bovine gelatin, type B, 225 bloom | GELITA USA, Inc. Sergeant Bluff, IA | gelatin |

### EXAMPLES

### Example 1. Crosslinked Chitosan Capsule With Isocyanate and Acrylate Crosslinkers

A chitosan stock solution is prepared by dispersing 121.50 g chitosan ChitoClear into 2578.5 g deionized water while mixing in a jacketed reactor. The pH of the chitosan dispersion is then adjusted to 5.12 using 48.60 g concentrated HCl under agitation. The temperature of the chitosan solution is then increased to 85° C. over 60 minutes and then held at 85° C. for a period of time to hydrolyze the ChitoClear. The temperature is then reduced to 25° C. after the hydrolyzing step over a period of 90 minutes. The pH of the hydrolyzed chitosan solution is 5.28. The formed chitosan stock solution was used for preparation of crosslinked chitosan capsule with isocyanate and acrylate in Example 1, 2, 8 and 9.

A water phase is prepared by mixing 308.70 g of the above chitosan stock solution in a jacketed reactor. An oil phase is prepared by mixing 102.64 g perfume and 25.66 g isopropyl myristate together along with 2.80 g Takenate D-110N at room temperature. The oil phase is added to the water phase under high shear milling to obtain an emulsion with desired particle size. The emulsion is heated to 70° C. A second acrylate crosslinker, 7.21 g trimethylolpropane triacrylate was then added to the above emulsion slowly under mixing. The obtained emulsion is then heated to 90° C. in 60 minutes and maintained at this temperature for 8 hours while mixing. The formed capsules have a median particle size of 43.80 microns. The capsules formed had a free oil of 0.19% and a one-week leakage of 14.20%.

### Example 2. Crosslinked Chitosan Capsule With Isocyanate and Acrylate Crosslinkers

A water phase is prepared by mixing 308.70 g of the above chitosan stock solution in a jacketed reactor. An oil phase is prepared by mixing 102.64 g perfume and 25.66 g isopropyl myristate together along with 2.80 g Takenate D-110N at room temperature. The oil phase is added to the water phase under high shear milling to obtain an emulsion with desired particle size. The emulsion is heated to 70° C. A second acrylate crosslinker, 10.82 g trimethylolpropane triacrylate was then added to the above emulsion slowly under mixing. The obtained emulsion is then heated to 90° C. in 60 minutes and maintained at this temperature for 8 hours while mixing. The formed capsules have a median particle size of 38.80 microns. The capsules formed had a free oil of 0.28% and a one-week leakage of 14.94%.

### Example 3. Crosslinked Gelatin Capsule With Isocyanate and Acrylate Crosslinkers

A water phase is prepared by dissolving 11.97 g type B Bovine gelatin with 225 bloom in 187.60 g deionized water while mixing in a jacketed reactor at 50° C. The water phase was then cooled down to 25° C. after gelatin was dissolved. An oil phase is prepared by mixing 102.64 g perfume and 25.66 g isopropyl myristate together along with 2.80 g Takenate D-110N at room temperature. The oil phase is added to the water phase under high shear milling to obtain an emulsion with desired particle size. The emulsion is heated to 70° C. A second acrylate crosslinker, 7.21 g trimethylolpropane triacrylate was then added to the above emulsion slowly under mixing. The obtained emulsion is then heated to 90° C. in 60 minutes and maintained at this temperature for 8 hours while mixing. The formed capsules have a median particle size of 20.54 microns. The capsules formed had a free oil of 0.04% and a one-week leakage of 8.24%.

### Example 4. Crosslinked Gelatin Capsule With Isocyanate and Acrylate Crosslinkers

A water phase is prepared by dissolving 11.97 g type B Bovine gelatin with 225 bloom in 187.60 g deionized water while mixing in a jacketed reactor at 50° C. The water phase was then cooled down to 25° C. after gelatin was dissolved. An oil phase is prepared by mixing 102.64 g perfume and 25.66 g isopropyl myristate together along with 2.80 g Takenate D-110N at room temperature. The oil phase is added to the water phase under high shear milling to obtain an emulsion with desired particle size. The emulsion is heated to 70° C. A second acrylate crosslinkers, 3.61 g trimethylolpropane triacrylate and 5.25 g CD9055 from Sartomer were then added to the above emulsion slowly under mixing. The obtained emulsion is then heated to 90° C. in 60 minutes and maintained at this temperature for 8 hours while mixing. The formed capsules have a median particle size of 16.83 microns. The capsules formed had a free oil of 0.15% and a one-week leakage of 52.98%.

### Example 5. Crosslinked Gelatin Capsule With Isocyanate and Acrylate Crosslinkers

A water phase is prepared by dissolving 11.97 g type B Bovine gelatin with 225 bloom in 227.50 g deionized water while mixing in a jacketed reactor at 50° C. The water phase was then cooled down to 25° C. after gelatin was dissolved. An oil phase is prepared by mixing 102.64 g perfume and 25.66 g isopropyl myristate together along with 2.80 g Takenate D-110N at room temperature. The oil phase is added to the water phase under high shear milling to obtain an emulsion with desired particle size. The emulsion is heated to 70° C. A second acrylate crosslinkers, 3.61 g trimethylolpropane triacrylate and 8.75 g 80% [2-(acryloyloxy)ethyl] trimethylammonium chloride solution were then added to the above emulsion slowly under mixing. The obtained emulsion is then heated to 90° C. in 60 minutes and maintained at this temperature for 8 hours while mixing. The formed capsules have a median particle size of 40.02 microns. The capsules formed had a free oil of 0.09% and a one-week leakage of 4.86%.

### Example 6. Crosslinked Gelatin Capsule With Isocyanate and Acrylate Crosslinkers

A gelatin solution modified with cationic acrylate was prepared by mixing 40.35 g Bovine gelatin, type B, 225 bloom, with 32.04 g 80% [2-(acryloyloxy) ethyl] trimethylammonium chloride solution in 600 g deionized water at 70° C. for 12 hours.

A water phase is prepared by mixing 210 g of the above gelatin solution modified with cationic acrylate in a jacket reactor at 25° C. An oil phase is prepared by mixing 102.64 g perfume and 25.66 g isopropyl myristate together along with 2.80 g Takenate D-110N at room temperature. The oil phase is added to the water phase under high shear milling to obtain an emulsion with desired particle size. The emulsion is heated to 70° C. A second acrylate crosslinker, 4.20 g trimethylolpropane triacrylate was then added to the above emulsion slowly under mixing. The obtained emulsion is then heated to 90° C. in 60 minutes and maintained at this temperature for 8 hours while mixing. The formed capsules have a median particle size of 16.06 microns. The capsules formed had a free oil of 0.14% and a one-week leakage of 30.16%.

### Example 7. Crosslinked Gelatin Capsule With Isocyanate and Acrylate Crosslinkers

A gelatin solution modified with anionic acrylate was prepared by mixing 39.48 g Bovine gelatin, type B, 225 bloom, with 18.66 g CD9055 acrylate from Sartomer in 600 g deionized water at 70° C. for 12 hours.

A water phase is prepared by mixing 210 g of the above gelatin solution modified with anionic acrylate in a jacket reactor at 25° C. An oil phase is prepared by mixing 102.64 g perfume and 25.66 g isopropyl myristate together along with 2.80 g Takenate D-110N at room temperature. The oil phase is added to the water phase under high shear milling to obtain an emulsion with desired particle size. The emulsion is heated to 70° C. A second acrylate crosslinker, 4.20 g trimethylolpropane triacrylate was then added to the above emulsion slowly under mixing. The obtained emulsion is then heated to 90° C. in 60 minutes and maintained at this temperature for 8 hours while mixing. The formed capsules have a median particle size of 43.43 microns. The capsules formed had a free oil of 0.09% and a one-week leakage of 44.77%.

### Example 8. Crosslinked Chitosan Capsule With Oil Phase Acrylate and Water Phase Acrylate Crosslinkers

A water phase is prepared by mixing 234.60 g of the chitosan stock solution from Example 1 with 108.00 g deionized water, and 3.46 g of 5% Selvol 540 at 70° C. An oil phase is prepared by mixing 66.59 g perfume and 54.48 g isopropyl myristate together along with 8.82 g SR368 from Sartomer at 70° C. in a jacketed reactor. The water phase is added to the oil phase without mixing at 70° C. A high shear was then applied to the mixture after all water phase was added to obtain an emulsion with desired particle size. A second acrylate crosslinker, 6.18 g trimethylolpropane triacrylate was then added to the above emulsion slowly under mixing. The obtained emulsion is then heated to 90° C. in 60 minutes and maintained at this temperature for 8 hours while mixing. The formed capsules have a median particle size of 32.11 microns. The capsules formed had a free oil of 0.16% and a one-week leakage of 26.31%.

### Example 9. Crosslinked Chitosan Capsule With Oil Phase Acrylate and Water Phase Acrylate Crosslinkers

A water phase is prepared by mixing 234.60 g of the chitosan stock solution from Example 1 with 108.00 g deionized water, and 6.96 g of 5 % Selvol 540 at 70° C. in a jacketed reactor. An oil phase is prepared by mixing 66.59 g perfume and 54.48 g isopropyl myristate together along with 7.26 g CN975 from Sartomer at 70° C. The oil phase is added to the water phase under high shear milling to obtain an emulsion with desired particle size. A second acrylate crosslinker, 6.18 g trimethylolpropane triacrylate was then added to the above emulsion slowly under mixing. The obtained emulsion is then heated to 90° C. in 60 minutes and maintained at this temperature for 8 hours while mixing. The formed capsules have a median particle size of 28.84 microns. The capsules formed had a free oil of 0.27% and a one-week leakage of 18.90%.

### Example 10. Crosslinked Gelatin Capsule With Oil Phase Acrylate and Water Phase Acrylate Crosslinkers

A gelatin solution is prepared by dissolving 20.58 g type B Bovine gelatin with 225 bloom in 210.00 g deionized water under mixing in a jacketed reactor at 50° C. A water phase is prepared by adding 4.90 g 5% Selvol 540 solution to the above gelatin solution at 25° C. An oil phase is prepared by mixing 64.16 g perfume and 64.16 g isopropyl myristate together along with 7.21 g CN975 from Sartomer at 70° C. The oil phase is added to the water phase under high shear milling to obtain an emulsion with desired particle size. A second acrylate crosslinker, 7.21 g trimethylolpropane triacrylate was then added to the above emulsion slowly under mixing. The obtained emulsion is then heated to 90° C. in 60 minutes and maintained at this temperature for 8 hours while mixing. The formed capsules have a median particle size of 22.82 microns. The capsules formed had a free oil of 0.99% and a one-week leakage of 77.55%.

### Example 11. Crosslinked Gelatin Capsule With Oil Phase Acrylate and Water Phase Acrylate Crosslinkers

A gelatin solution is prepared by dissolving 20.58 g type B Bovine gelatin with 225 bloom in 210.00 g deionized water under mixing in a jacketed reactor at 50° C. A water phase is prepared by adding 4.90 g 5% Selvol 540 solution to the above gelatin solution at 25° C. An oil phase is prepared by mixing 64.16 g perfume and 64.16 g isopropyl myristate together along with 7.21 g CN975 from Sartomer at 70° C. The oil phase is added to the water phase under high shear milling to obtain an emulsion with desired particle size. A second and a third acrylate crosslinkers, 3.64 g trimethylolpropane triacrylate and 5.14 g tetra (ethylene glycol) diacrylate were then added to the above emulsion slowly under mixing. The obtained emulsion is then heated to 90° C. in 60 minutes and maintained at this temperature for 8 hours while mixing. The formed capsules have a median particle size of 23.36 microns. The capsules formed had a free oil of 0.25% and a one-week leakage of 67.12%.

### Example 12. Crosslinked Gelatin Capsule With Oil Phase Acrylate and Water Phase Acrylate Crosslinkers

A water phase is prepared by dissolving 20.58 g type B Bovine gelatin with 225 bloom in 210.00 g deionized water under mixing in a jacketed reactor at 50° C. The water phase is then cooled down to 25° C. after gelatin was dissolved. An oil phase is prepared by mixing 64.16 g perfume and 64.16 g isopropyl myristate together along with 7.21 g CN975 from Sartomer at 70° C. The oil phase is added to the water phase under high shear milling to obtain an emulsion with desired particle size. A second and a third acrylate crosslinkers, 3.64 g trimethylolpropane triacrylate and 5.14 g tetra (ethylene glycol) diacrylate were then added to the above emulsion slowly under mixing. The obtained emulsion is then heated to 90° C. in 60 minutes and maintained at this temperature for 8 hours while mixing. The formed capsules have a median particle size of 35.60 microns. The capsules formed had a free oil of 0.15% and a one-week leakage of 66.67%.

Percent degradation is measured according to the OECD Guidelines for the Testing of Chemicals, test method OECD 301B. A copy is available in www.oecd-ilibrary.org.

Capsules according to the invention can have core to wall ratios even as high as 95% core to 1% wall by weight. In applications where enhanced degradability is desired, higher core to wall ratios can be used such as 99% core to 1% wall, or even 99.5% to 0.5% by weight or higher. With appropriate selection of core to wall ratios, the shell of the composition according to the invention can be selected to achieve a % degradation of at least 40% degradation after 14 days, of at least 50% degradation after at least 20 days, and of at least 60% degradation after at least 28 days when tested according to test method OECD 301B.

Uses of singular “a,” “an,” are intended to cover both the singular and the plural, unless otherwise indicated herein or clearly contradicted by context. The terms “comprising,” “having,” “including,” and “containing” are to be construed as open-ended terms. All references, including publications, patent applications, and patents, cited herein are hereby incorporated by reference. Any description of certain embodiments as “preferred” embodiments, and other recitation of embodiments, features, or ranges as being preferred, or suggestion that such are preferred, is not deemed to be limiting. The invention is deemed to encompass embodiments that are presently deemed to be less preferred and that may be described herein as such. All methods described herein can be performed in any suitable order unless otherwise indicated herein or otherwise clearly contradicted by context. The use of any and all examples, or exemplary language (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims.

What is claimed is:

1. A delivery particle comprising a core material and a shell encapsulating the core material, wherein the core material comprises a benefit agent; and,
wherein the shell comprises a polymer, the polymer comprising the reaction product of:
an isocyanate or acid chloride or oil soluble bi- or multi-functional (meth)acrylate with an amine-containing natural material having free amino moieties, and
an α, β -unsaturated compound, the α, β -unsaturated compound forming C-N covalent bonds with the amine moieties of the natural material,
the % wt ratio of the isocyanate to amine-containing natural material to α, β -unsaturated compound being in the range from 0.1:90:9.9 to 20:10:70 based on weight of the polymer.

2. The delivery particle according to claim 1 wherein the α, β -unsaturated compound forms C-N covalent bonds,
wherein the natural material is selected from chitosan, chitin, gelatin, amine containing starch, amino sugar, polylysine, or hyaluronic acid; and
wherein the α, β -unsaturated compound is selected from a water-soluble or dispersible acrylate, an alkyl acrylate, an α, β -unsaturated ester, an acrylic acid, an acrylamide, a vinyl ketone, a vinyl sulfone, a vinyl phosphonate, an acrylonitrile derivative or mixtures thereof.

3. The delivery particle according to claim 2, wherein the α, β -unsaturated compound is a monofunctional, bifunctional, or multifunctional polymeric compound or mixtures thereof.

4. The delivery particle according to claim 2, wherein the α, β -unsaturated compound is selected from acrylamide, methacrylamide, n-isopropyl acrylamide, (3-acrylamidopropyl) trimethylammonium chloride, or 2-acrylamido-2-methyl-1-propanesulfonic acid.

5. The delivery particle according to claim 1, wherein the α, β -unsaturated compound is anionic charged.

6. The delivery particle according to claim 1, wherein the α, β -unsaturated compound is cationic charged.

7. The delivery particle according to claim 1, wherein a zeta potential of the delivery particle is -100 mV - +200 mV at pH 3 and -200 mV - +100 mV at pH 10. The delivery particle of claim 1, wherein, in addition, a portion of the free amino moieties of the natural material are reacted with a α, β -unsaturated compound via an Aza-Michael Addition reaction.

8. The delivery particle of claim 1, wherein, in addition, a portion of the free amino moieties of the natural material are reacted with an isocyanate, acid chloride, or acrylate to form a urea, amide, or an amino ester bond respectively.

9. The delivery particle of claim 8, wherein the isocyanate is selected from the group consisting of a polyisocyanurate of toluene diisocyanate, a trimethylol propane adduct of toluene diisocyanate, a trimethylol propane adduct of xylylene diisocyanate, methylene diphenyl isocyanate, toluene diisocyanate, tetramethylxylidene diisocyanate, naphthalene-1,5-diisocyanate, and phenylene diisocyanate.

10. The delivery particle of claim 8, wherein the acid chloride is selected from terephthaloyl chloride, isophthaloyl chloride, phthaloyl chloride, 1,3,5-benzenetricarbonyl trichloride, adipoyl chloride, glutaryl chloride, or sebacoyl chloride.

11. The delivery particle of claim 1, wherein the oil soluble (meth)acrylate is selected from group consisting of a bi-functional (meth)acrylate, a tri-functional (meth)acrylate, a tetra-functional (meth)acrylate, a penta-functional (meth)acrylate, a hexa-functional (meth)acrylate, a hepta-functional (meth)acrylate, an octa-functional (meth)acrylate and mixtures thereof.

12. The delivery particle of claim 2, wherein the water soluble or dispersible (meth)acrylate is selected from 2-carboxyethyl acrylate, 2-carboxyethyl acrylate oligomers, 2-carboxypropyl acrylate, 4-acryloyloxyphenylacetic acid, carboxyoctyl acrylate, tripropylene glycol diacrylate, ethoxylated bisphenol diacrylate, dipropylene glycol diacrylate, alkoxylated hexanediol diacrylate, alkoxylated cyclohexane dimethanol diacrylate, propoxylated neopentyl glycol diacrylate, trimethylolpropane triacrylate, pentaerythritol triacrylate, ethoxylated trimethylolpropane triacrylate, propoxylated trimethylolpropane triacrylate, propoxylated glyceryl triacrylate, ditrimethylolpropane tetraacrylate, dipentaerythritol pentaacrylate, ethoxylated pentaerythritol tetraacrylate, glycerol tri(meth)acrylate, ethylene glycol diacrylate, di-, tri-, tetra-, or pentaethylene glycol diacrylate, dipropylene glycol diacrylate, polyethylene glycol diacrylate, 2-ethylhexyl acrylate, 2-hydroxyethyl (meth)acrylate, cyanoethyl acrylate, 2-hydroxypropyl acrylate, lauryl acrylate, cyclohexyl acrylate, tetrahydrofurfuryl acrylate, chlorobenzyl acrylate, amino alkylacrylate, ethylaminoethyl (meth)acrylate, aminoethyl (meth)acrylate, tertiarybutyl aminoethyl (meth)acrylate, diethylamino (meth)acrylate, diethylaminoethyl (meth)acrylate, dimethylaminoethyl (meth)acrylate independently or a combination of the foregoing.

13. The delivery particle of claim 1, wherein the benefit agent is a fragrance, preferably a fragrance comprising perfume raw materials characterized by a logP of from about 2.5 to about 4.5.

14. The delivery particle of claim 1, wherein the core comprises in addition a partitioning modifier selected from the group consisting of isopropyl myristate, vegetable oil, modified vegetable oil, mono-, di-, and tri-esters of C4-C24 fatty acids, dodecanophenone, lauryl laurate, methyl behenate, methyl laurate, methyl palmitate, methyl stearate, and mixtures thereof, preferably isopropyl myristate.

15. The delivery particle of claim 1, wherein the wall has a biodegradability above 30% CO2 in 60 days following OECD 301B test, preferably above 40% CO2, more preferably above 50% CO2, even more preferably above 60% CO2 (maximum 95%).

16. The delivery particle according to claim 1, wherein the wall of the delivery particles further comprises a coating material, preferably wherein the coating material is selected from the group consisting of poly(meth)acrylate, poly(ethylene-maleic anhydride), polyamine, wax, polyvinylpyrrolidone, polyvinylpyrrolidone co-polymers, polyvinylpyrrolidone-ethyl acrylate, polyvinylpyrrolidone- vinyl acrylate, polyvinylpyrrolidone methacrylate, polyvinylpyrrolidone/vinyl acetate, polyvinyl acetal, polyvinyl butyral, polysiloxane, poly(propylene maleic anhydride), maleic anhydride derivatives, co-polymers of maleic anhydride derivatives, polyvinyl alcohol, styrenebutadiene latex, gelatine, gum arabic, carboxymethyl cellulose, carboxymethyl hydroxyethyl cellulose, hydroxyethyl cellulose, other modified celluloses, sodium alginate, chitosan, chitin, casein, pectin, modified starch, polyvinyl acetal, polyvinyl butyral, polyvinyl methyl ether/maleic anhydride, polyvinyl pyrrolidone and its co polymers, poly(vinyl pyrrolidone/methacrylamidopropyl trimethyl ammonium chloride), polyvinylpyrrolidone/vinyl acetate, polyvinyl pyrrolidone/dimethylaminoethyl methacrylate, polyvinyl amines, polyvinyl formamides, polyallyl amines, copolymers of polyvinyl amines, and mixtures thereof.

17. The delivery particle according to claim 1, wherein the delivery particle has a leakage of below about 50%, or at most about 50% as determined by the Leakage Test described in the TEST METHODS Section.

18. The delivery particle according to claim 1 wherein the delivery particle has a volume weighted median particle size of from 5 microns to 150 microns, or even from 10 to 50 microns, or even from 15 to 50 microns.

19. A process of forming a population of delivery particles, the delivery particles comprising a core material and a shell encapsulating the core material, wherein the core material comprises a benefit agent; and, wherein the shell comprises a polymer,
the polymer comprising the reaction product of:
i) an isocyanate or acid chloride or bi- or multi-functional (meth)acrylate with
ii) an amine-containing natural material having free amino moieties, and
iii) an α, β -unsaturated compound,
the process comprising:
i)forming a water phase comprising dissolving or dispersing in water an amine-containing natural material;
ii)forming an oil phase by mixing together a benefit agent, preferably perfume, optionally a partitioning modifier, and optionally a solvent, together with a shell-forming materials selected from the group consisting of an isocyanate, an acid chloride, and an oil-soluble bi- or multi- functional (meth)acrylate;
iii)emulsifying the oil phase into the water phase to form an emulsion and heating the emulsion to initiate formation of a polyurea, polyamide, or polyaminoester shell between the free amino moieties on the natural polymer and the isocyanate, the acid chloride, or multi-functional(meth)acrylate respectively;
iv)adding to the emulsion with mixing, grinding or heating, while the shell is forming, an α, β -unsaturated compound comprising a water-soluble or dispersible acrylate, an alkyl acrylate, an α, β -unsaturated ester, an acrylic acid, an acrylamide, a vinyl ketone, a vinyl sulfone, a vinyl phosphonate, or an acrylonitrile derivative,
the α, β -unsaturated compound thereby forming C-N covalent bonds with a portion of the amine groups of the natural material.

20. An article of manufacture incorporating the delivery particles according to claim 1.

21. The article of manufacture according to claim 20 wherein the article is selected from the group consisting of an agricultural formulation, a slurry encapsulating an agricultural active, a population of dry microcapsules encapsulating an agricultural active, an agricultural formulation encapsulating an insecticide, and an agricultural formulation for delivering a preemergent herbicide.

22. The article of manufacture according to claim 20 wherein the agricultural active is selected from the group consisting of an agricultural herbicide, an agricultural pheromone, an agricultural pesticide, an agricultural nutrient, an insect control agent and a plant stimulant.
